(19) United States
(12) Patent Application Publication
(10) Pub. No.: US 2016/0203288 A1
(43) Pub. Date: Jul. 14, 2016

(54) Systems and Methods for Identifying Historical Vasculature Cases

(71) Applicant: The Research Foundation for the State University of New York, Amherst, NY (US)

(72) Inventors: Hui Meng, Buffalo, NY (US); Luca Antiga, Bergamo (IT); Jianping Xiang, Buffalo, NY (US)

(21) Appl. No.: 14/408,195

(22) PCT Filed: Jun. 18, 2013
(86) PCT No.: PCT/US13/46423

§ 371 Date: Dec. 15, 2014

Related U.S. Application Data
(60) Provisional application No. 61/661,043, filed on Jun. 18, 2012.

(51) Int. Cl. G06F 19/00; G06F 17/10; G06T 7/60; A61B 5/02; G06T 7/00

(52) U.S. Cl. CPC G06F 19/3437; A61B 5/02007; G06T 7/0081; G06T 7/60; G06T 7/0012; G06F 17/10; G06T 2207/30104

(57) ABSTRACT

Systems and methods for identifying historical vasculature cases are disclosed. A method may use a case database and a processor. One such method comprises the step of retrieving an image of the vasculature, demographic factors, morphological factors, and hemodynamic factors of the individual. The image is segmented and a geometry is calculated using the processor. Fluid flow may be simulated based on the hemodynamic factors and the calculated geometry. Calculations and factors are stored in the case database and relevant cases are identified based on the calculated geometry of the vasculature, the simulated fluid flow, and the demographic factors in comparison to the cases in the case database.

### FIELD OF THE INVENTION

The invention relates to systems and methods for identifying historical vasculature cases. For example, identifying historical vasculature cases that resemble or are similar to a vasculature of an individual.

### BACKGROUND OF THE INVENTION

Neurointerventionists are routinely faced with the dilemma of whether or not to treat a vasculature of an individual. One particularly troublesome condition is an unruptured intracranial aneurysm (“IA”). Vascular intracranial aneurysms widely affect up to 5% of the entire population. Aneurysm rupture leads to subarachnoid hemorrhage (SAH), a devastating event with high morbidity and mortality. With advancements in neurovascular imaging technology, incidental findings of unruptured intracranial aneurysms have become increasingly common.

While only 1% of aneurysms rupture, the decision to treat an aneurysm is difficult, since treatment holds a high risk of complications. Although the morbidity and mortality associated with rupture may suggest that an incidentally detected aneurysm should be treated to forestall the catastrophic event of SAH, the two current methods of treatment (open microsurgical aneurysm clip ligation or endovascular aneurysm coil embolization) are not without some risk of major morbidity and mortality. The daily dilemma of whether or not to treat asymptomatic unruptured aneurysms has created a need for tools to help in this decision making process.

Rupture risk prediction models should theoretically be built from longitudinal aneurysm data. However, most diagnosed unruptured intracranial aneurysms (“UIAs”) are treated, leaving only a small number of UIAs for conservative observation and periodical imaging. These cases tend to involve aneurysms small in size with low assumed rupture risks, or aneurysms having no clear surgical options, or patients who refuse treatment. Therefore, models built from longitudinal data tend to be severely skewed, not accounting for all of high-risk aneurysm features.

Recently, advancements in image-based computational fluid dynamics simulations and morphological analysis have helped researchers to stratify aneurysm rupture risk. Evidence from in vitro and in vivo studies has demonstrated that vascular hemodynamics plays a fundamental role in the outcome of an aneurysm. The significance of hemodynamic and morphological factors in aneurysm evaluation has increasingly gained clinical awareness, as reflected by a recently updated guideline for intracranial aneurysm management by the American Heart/Stroke Association.

Historically, engineers would run computer simulations in order to assist medical researchers examine rupture risk. However, these simulations were difficult to modify, lengthy in computational power required, and of limited relevance to clinicians.

Therefore, an accurate metric (or several metrics) to judge the risk of rupture of an aneurysm is critical to aid in generating the best possible treatment algorithm. Past studies to associate hemodynamics with aneurysm rupture status have focused on qualitative descriptions, in which complex flow patterns and multiple vortices have been associated with ruptured aneurysms. Complex flow patterns have been thought to increase inflammatory cell infiltration in the aneurysm wall, thereby increasing rupture risk. However, these qualitative descriptions are incomplete.

Several past studies have investigated potential metrics, however, they lack reliability and fail to capture the holistic condition of the aneurysm in the individual. For example, one metric is IA size. Although aneurysms exceeding 10 mm in size are considered to be dangerous, several studies have shown that a large percentage of ruptured aneurysms are, in fact, smaller than 10 mm. As such, this metric—although helpful—is rather unreliable.

Aneurysm shape has been studied as well, and certain shape parameters show stronger correlation with rupture than IA size. Aspect ratio (“AR”), defined as IA height divided by neck diameter, is the most commonly studied shape parameter. Although most findings affirm its importance, they do not converge on a common threshold value. Other studies have also investigated additional factors that correlate with IA rupture risk, however, these studies have not yielded quantifiable metrics that can be readily integrated into the clinical decision-making process. Adding complexity from such diverse variables into our current study would make risk assessment analysis unwieldy. Furthermore, these proposed indicators are not relevant to clinicians when presented without context.

Furthermore, current methods of assisting clinicians in treatment decisions critically depend on accurate 3-dimensional images. These methods present a calculated probability of aneurysm rupture, but the calculations may be subject to a margin of error because of poor or incomplete imaging. In turn, clinicians lack trust in the summary diagnosis of current methods and tend to rely upon their own experience to guide treatment decisions.

### BRIEF SUMMARY OF THE INVENTION

The systems and methods of the present disclosure are based on a comprehensive examination of morphology-based, hemodynamics-based, and demographics-based factors from a cohort of ruptured and unruptured IA cases. Instead of presenting a clinician with an opaque and abstract calculation of rupture probability, the present systems and methods identify relevant historical vasculature cases for the clinician and allow the clinician to augment their experience when making treatment decisions. Further, the systems and methods of the present invention encompass quantitative and qualitative metrics that can be used to assess IAs in clinical settings.

The present invention can be described as a method of identifying historical vasculature cases that resemble a vasculature of an individual. The method involves using a case database which comprises historical vasculature cases. The method also involves using a processor in electronic communication with the case database.

The method comprises the step of retrieving an electronic image of the vasculature of the individual. The step of retrieving an image of the vasculature of the individual may comprise the sub-steps of using a detector to capture an image of the vasculature of the individual and storing the image of the vasculature of the individual such that the vascular image is electronically associated with the individual. The image of the vasculature may be stored in an electronic picture archiving and communication system (“PACS”). The image of the vasculature may also be stored in the case database.

The method further comprises the step of retrieving demographic factors and morphological (e.g., geometric) factors of the individual. The retrieving step may comprise the sub-steps of using an input device to input the demographic factors and morphological factors of the individual and storing the demographic factors and morphological factors of the individual such that the demographic factors and morphological factors are associated with the individual. The demographic factors, morphological factors, and hemodynamic factors of the individual may be stored in the case database. The input device may be a laptop, mobile phone, workstation, tablet, front-end computer, and/or any other suitable input device modality.

The method further comprises the step of using the processor to segment the retrieved image. The image may be segmented on a branch-by-branch basis. In one embodiment the step of segmenting the retrieved image comprises the sub-steps of using an input device to place seed spheres along a branch of interest and identifying a corresponding branch name.

In one embodiment, the method further comprises the step of defining, using an input device, a neck plane, region of interest, and/or a main inlet parent vessel. In another embodiment, segmenting the retrieved image is based on the defined neck plane, region of interest, and/or main inlet parent vessel.

The method further comprises the step of using the processor to calculate a geometry of the vasculature based on the segmented image.

The method may further comprise the step of storing the calculated geometry of the vasculature in the historical vasculature case database. The demographic factors and morphological factors of the individual may also be stored in the historical vasculature case database.

The method further comprises the step of using the processor to identify historical vasculature cases of the case database that resemble the vasculature of the individual. The identification step is performed by the processor based on the calculated geometry of the vasculature and the morphological factors of the historical vasculature cases of the case database. The identification step is also based on the demographic factors of the individual and the demographic factors of the historical vasculature cases of the case database. The cases of the case database may be identified for the purpose of clinical intervention and/or the purpose of building a rupture resemblance scale database.

In one embodiment, the method further comprises the step of using the processor to calculate, for each identified historical vasculature case a morphological resemblance value and a demographic resemblance value. The morphological resemblance value is based on the calculated geometry of the vasculature and the identified historical vasculature case. The demographic resemblance value is based on the demographic factors of the individual and the identified historical vasculature case.

In another embodiment, the method further comprises the step of determining an aneurysm rupture resemblance scale or aneurysm rupture risk for the vasculature of the individual based on the calculated morphological resemblance values and calculated demographic resemblance values.

In one embodiment, the method further comprises the steps of using the input device to adjust the geometry of the vasculature and re-calculating the calculated geometry of the vasculature. The geometry of the vasculature may be adjusted to account for future clinical intervention and/or to account for a future surgical device.

The invention may also be described as a method of identifying historical vasculature cases that resemble a vasculature of an individual, wherein hemodynamics are analyzed. These methods use a case database comprising historical vasculature cases and a processor in electronic communication with the case database. In one embodiment, the method comprises the steps of retrieving an image of the vasculature of the individual and retrieving demographic factors, morphological factors, and hemodynamic factors of the individual. Hemodynamic factors may include blood viscosity and blood density values of the individual. The processor is used to segment the retrieved image. The processor is also used to calculate a geometry of the vasculature based on the segmented image.

The method further comprises the step of using the processor to simulate fluid flow in the vasculature based on the hemodynamic factors associated with the individual and the calculated geometry of the vasculature. In one embodiment, the accuracy and/or speed of simulating fluid flow in the vasculature can be configured using an input device.

The method may further comprise the step of storing the calculated geometry of the vasculature, simulated fluid flow of the vasculature, demographic factors, morphological factors, and hemodynamic factors of the individual in the historical vasculature case database.

The method further comprises using the processor to identify historical vasculature cases of the case database that resemble the vasculature of the individual. The identification may be based on the calculated geometry of the vasculature and the morphological factors of the historical vasculature cases of the case database. The identification may also be based on the simulated fluid flow in the vasculature and the hemodynamic factors of the historical vasculature cases of the case database. The identification may also be based on the demographic factors of the individual and the demographic factors of the historical vasculature cases of the case database.

In one embodiment, the method further comprises the step of using a display to visualize the simulated fluid flow in the vasculature.

In another embodiment, the method further comprises the step of using the processor to calculate, for each identified historical vasculature case a morphological resemblance value, a demographic resemblance value, and a hemodynamic resemblance value. The morphological resemblance value may be based on the calculated geometry of the vasculature and the identified historical vasculature case. The demographic resemblance value may be based on the demographic factors of the individual and the identified historical vasculature case. And, the hemodynamic resemblance value may be based on the simulated fluid flow in the vasculature and the identified historical vasculature case.

In one embodiment, the method may further comprise the step of determining an aneurysm rupture resemblance scale and/or an aneurysm rupture risk for the vasculature of the individual based on the calculated hemodynamic resemblance values, calculated morphological resemblance values, and calculated demographic resemblance values.

In another embodiment, the method may further comprise the steps of adjusting the geometrical parameters of the vasculature, re-calculating the calculated geometry of the vasculature of the individual, and re-simulating the fluid flow in the vasculature based on the re-calculated geometry of the vasculature.

The present invention may also be described as a system for identifying historical vasculature cases that resemble a vasculature of an individual. The system comprises an input device, a case database, and a processor. The input device is configured to capture demographic factors and morphological factors of the individual. The case database is in electronic communication with the input device and configured to comprise historical vasculature cases.

The processor is in electronic communication with the case database and configured to retrieve an image of the vasculature of the individual, retrieve demographic factors, morphological factors, and hemodynamic (e.g., flow) factors of the individual, segment the retrieved image, calculate a geometry of the vasculature based on the segmented image, store the calculated geometry of the vasculature, demographic factors and morphological factors of the individual in the historical vasculature case database, and identify historical vasculature cases of the case database that resemble the vasculature of the individual. The identification may be based on the calculated geometry of the vasculature and the historical vasculature cases of the case database and the demographic factors of the individual and the historical vasculature cases of the case database

In one embodiment, the system further comprises a detector configured to capture an image of vasculature of the individual. The detector may be in electronic communication with the case database and the processor. The detector may be configured for digital subtraction angiography, computed tomography angiography, ultrasound, and/or magnetic resonance angiography.

In another embodiment, the processor is further configured to simulate fluid flow in the vasculature based on the hemodynamic factors associated with the individual and the calculated geometry of the vasculature and identify historical vasculature cases of the case database that resemble the vasculature of the individual based on the simulated fluid flow in the vasculature and the historical vasculature cases of the case database.

In one embodiment, the demographic factors may comprise an age, sex, race, education, blood pressure, health behavior, and/or family history of an individual. The morphological factors may comprise size, aneurysm height, aneurysm perpendicular height, aspect ratio, bottleneck factor, size ratio, aneurysm inclination angle, aneurysm volume to ostium area ratio, height to width ratio, undulation index, ellipticity index, non-spehericty index, and/or ostium ratio of an individual. The hemodynamic factors may comprise wall shear stress, oscillatory shear index, energy loss, pressure, pathlines, streaklines, streamlines, particle paths, inflow jet, impingement zone, and/or velocity.

In another embodiment, the system further comprises a display configured to simultaneously show two or more visualizations of the vasculature of the individual.

The systems and methods of the present invention may be configured to assist with aneurysm treatment decisions, especially for aneurysms with size less than 7 mm—the current threshold for treatment, or aneurysms with ambiguous prognoses. The present invention may aid treatment planning by visualizing flow directions, impingement, and flow jet location. For example, the present invention may assist clinicians with determining if an aneurysm should be treated by using stent-assisted coiling and where to locate such a stent.

The systems and methods of the present invention may be embodied as a software tool that can facilitate clinical analysis of morphological, hemodynamic, and demographic characteristics of cerebral aneurysms in order to aid treatment planning. As an image-based vascular analysis program, aneurysms can be rapidly evaluated using patient specific hemodynamic, morphologic, and demographic data. Detailed analyses of aneurysm morphology and hemodynamics are integrated into the clinical workflow and provide medical practitioners with unprecedented insights into the rupture disposition of the individual aneurysms that they face at the point of care.

For example, the clinical workflow of an exemplary embodiment of the present invention utilizes image data from a PACS and performs interactive image segmentation, automatic morphometric calculation, parallel-computing-based pulsatile hemodynamic analysis, rupture risk evaluation, and web-based data management. In time, this embodiment can provide analysis to every participating healthcare provider, in order to facilitate on-site decision-making, and furthermore, can contribute aneurysm cases to a central case database. The continuous growth of such a case database would further improve its representativeness of the entire aneurysm population, and greatly improve the accuracy of rupture evaluation.

### DESCRIPTION OF THE DRAWINGS

For a fuller understanding of the nature and objects of the invention, reference should be made to the following detailed description taken in conjunction with the accompanying drawings, in which:

FIG. 1 is a flowchart illustrating a method according to one embodiment of the present invention;

FIG. 2 is a flowchart illustrating another method according to one embodiment of the present invention;

FIG. 3 is a flowchart illustrating another method according to one embodiment of the present invention;

FIG. 4 is a flowchart illustrating another method according to one embodiment of the present invention;

FIG. 5 is a flowchart illustrating another method according to one embodiment of the present invention;

FIG. 6 is a diagram illustrating a system according to one embodiment of the present invention;

FIG. 7A is a diagram illustrating a graphical user interface presented to a clinician according to the disclosed invention;

FIG. 7B is a diagram illustrating a graphical user interface for vasculature visualization presented to a clinician according to the disclosed invention;

FIG. 8A is a diagram of a visualized vasculature having inscribed hemodynamic and morphological measurements; and

FIG. 8B is a diagram of a visualized vasculature having inscribed hemodynamic and morphological measurements.

### DETAILED DESCRIPTION OF THE INVENTION

The present invention provides systems and methods of identifying historical vasculature cases that resemble a vasculature of an individual. For example the vasculature of interest may be an unruptured aneurysm located in an individual, such as an intracranial aneurysm or abdominal aneurysm. A case database is configured to have historical vasculature cases for use by the system.

Exemplary Case Database

In one embodiment, historical vasculature cases may be stored in a relational database. The term historical includes vasculature cases that are concluded (e.g., through aneurysm rupture or intervention) and that are ongoing (e.g., unruptured aneurysms that are continuing to be monitored by a clinician). In a relational database, a column is a set of data values of a particular type, one for each row of the table. The term “field” may refer to the entire column, or a single item that exists at the intersection between one row and row column. A field may be configured to allow for various types of data including numbers, letters, free-form text, or predetermined values.

In another embodiment, the case database may be centralized and configured for anonymized data collection, statistical model building, and data mining. Portions of the case database may exist on electronic storage devices such as one or more computer hard drives. The hard drives may be located within a network-accessible server. In one embodiment, the electronic storage devices may be located in geographically diverse locations. For example, multiple medical institutions may have a server containing historical vasculature cases that were observed and treated at each medical institution. Each server would then comprise a portion of the case database, thereby allowing clinicians from one medical institution access to the historical vasculature data of other medical institutions.

Portions of the central case database may be mirrored and/or synchronized with child case databases. For example, each hospital may have a child case database which stores historical vasculature data for that particular hospital. The child case databases are synchronized with a parent case database thereby combining the historical vasculature data from every hospital into a central database.

Each historical vasculature case may comprise demographic, morphologic, and hemodynamic factors of the individual. These factors are explained in further detail below. Each vasculature case may also be associated with a medical institution and a supervising clinician, such that the vasculature cases may be organized and searched by medical institution name or supervising clinician name.

Exemplary Processor

For the purposes of the present invention, a processor may comprise one or more microprocessors working in parallel. For example, the processor may be embodied as a distributed computer running over a network (i.e., cloud computer). The processor may also be embodied as a high-performance device such as a supercomputer. As computer performance increases, the processor may be embodied in a personal computer workstation.

The processor is in electronic communication with the case database. For example, the processor may be in electronic communication with a local hard drive containing the case database. The processor may also be in electronic communication with a remote electronic storage device over a network, such as the Internet.

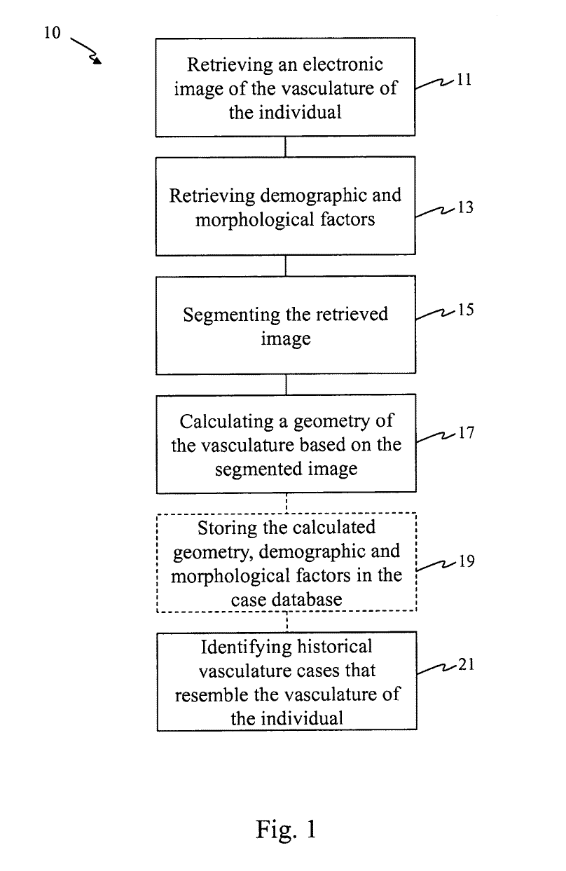

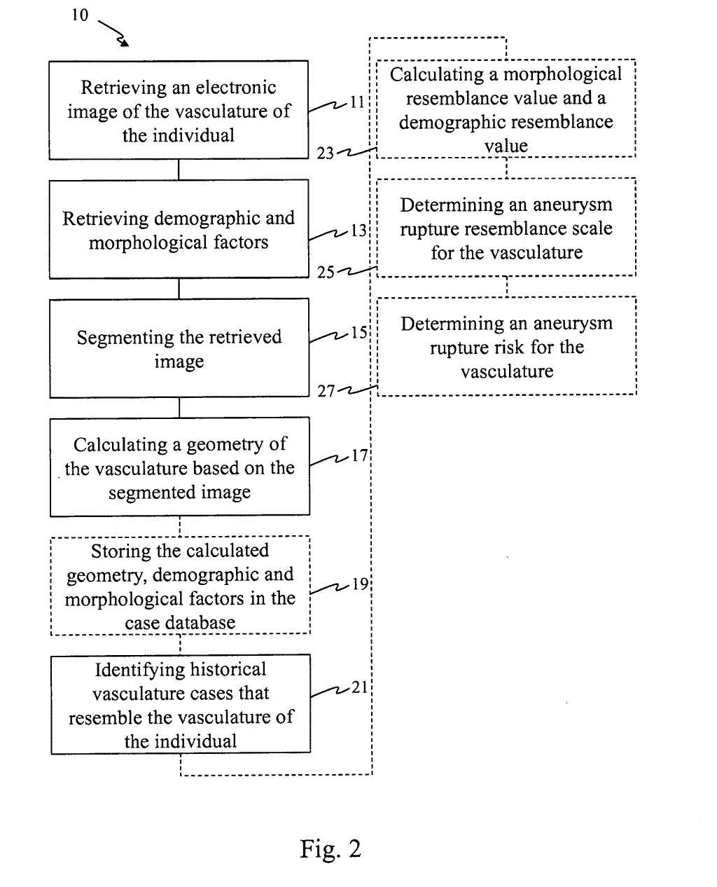

FIGS. 1-3 illustrate a method 10 according to the present invention. Method 10 may comprise the step of retrieving 11 an electronic image of the vasculature of the individual. The electronic image of the vasculature may be retrieved 11 from the case database or a PACS. For example, the processor may request the electronic image of the vasculature from an electronic storage device. In one embodiment, the electronic image is a Digital Imaging and Communications in Medicine image (“DICOM”). The electronic image may be embedded with information, such as the name of the individual.

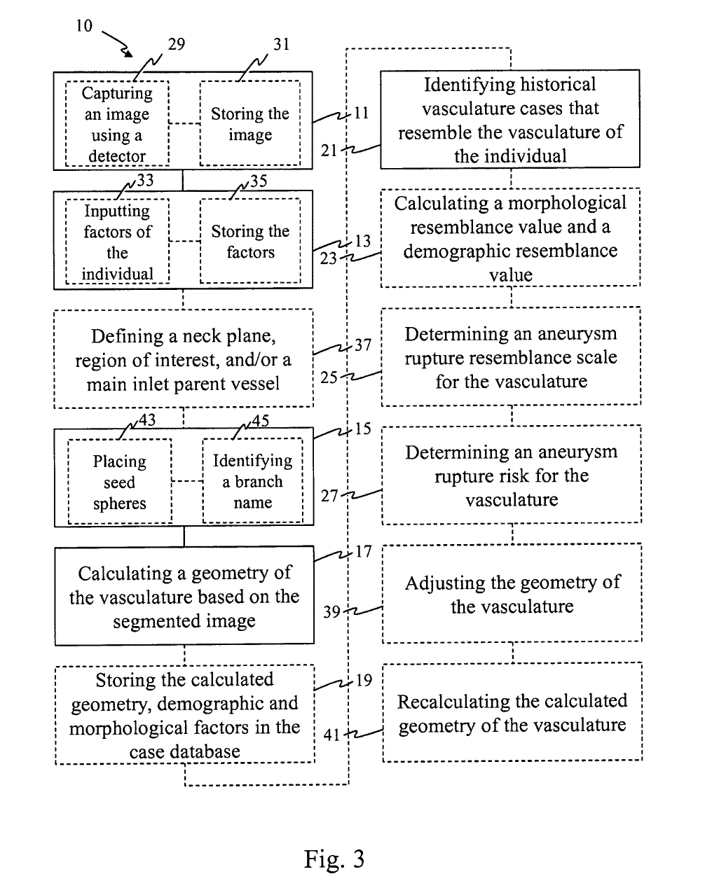

The step of retrieving 11 an image of the vasculature of the individual may comprise the sub-steps of using a detector to capture 29 an image of the vasculature of the individual and storing 31 the image of the vasculature of the individual such that the vascular image is electronically associated with the individual. One such method is illustrated in FIG. 3. The detector may be a device or procedure capable of capturing 29 an image of the vasculature of the individual such as magnetic resonance angiogram, computed tomography angiogram, digital subtraction angiogram, an ultrasound, or other imaging modalities known to be suitable for use with vasculature. The device may be capable of storing 31 the image directly to a PACS, the case database, or other storage locations. The image may be 2-dimension, 3-dimensional, or 4-dimensional (where the fourth dimension is time). The image may be a series of images or a composite image.

The method 10 may further comprise the step of retrieving 13 demographic factors and morphological factors of the individual. The demographic factors and morphological factors may be retrieved 13 from the case database or from a clinician. In one example, the clinician is presented with a clinical workflow interface on an input device (e.g., a laptop computer, tablet computer, mobile phone, or personal workstation). The clinical workflow interface may request demographic factors and morphological factors from the clinician. Once inputted 33 into the input device, the demographic and morphological factors of the individual may be stored 35. One such method is illustrated in FIG. 3.The demographic and morphological factors of the individual may be stored 35 such that the demographic factors and morphological factors are associated with the individual.

The method 10 may further comprise the step of segmenting 15 the retrieved image, using the processor. In one embodiment, segmentation 15 may be performed on a branch-by-branch basis as well as segmenting the aneurysm dome. Segmentation 15 may take place by placing 43 seed spheres along the branch of interest on a MIP or on slices and by identifying 45 the corresponding branch name from a pre-defined set. One such method is illustrated in FIG. 3 From the seeds, a deformable tubular model initializes and is evolved toward the lumen boundaries. Other embodiments of segmentation are known and are suitable for use in the present invention.

In one embodiment, the method 10 further comprises the step of defining 37, using an input device, a neck plane, region of interest, and/or a main inlet parent vessel. For example, the clinician may view the image on a display and use an input device to define 37 where the neck plane, region of interest, and/or main inlet parent vessel is located. In another embodiment, segmenting 15 the retrieved image is based on the defined neck plane, region of interest, and/or main inlet parent vessel. A possible neck plane, region of interest, and/or a main inlet parent vessel may be suggested to the clinician in order to simply the workflow for the clinician. The clinician may be able to use the input device to alter the suggestions or accept the suggestions as valid. Defining 37 the neck plane, region of interest, and/or a main inlet parent vessel provides important starting points for geometric calculations.

The method 10 may further comprise the step of calculating 17, using the processor, a geometry of the vasculature based on the segmented image. The geometry may be a mathematical representation of the vasculature. Calculating 17 the geometry may be an iterative process. In some embodiments, increasing the amount of calculations 17 will also will result in a more accurate the mathematical representation at the sacrifice of speed. In such embodiments, an accuracy of the calculated geometry may be configured such that the calculations 17 are performed more quickly. A calculated geometry of the vasculature allows for better modeling and more accurate comparison of the vasculature to historical vasculature cases.

In one embodiment, an intracranial aneurysm geometry is calculated 17. To calculate 17 the geometry, centerlines may first be generated from the common carotid artery (“CCA”) to each of the internal carotid artery (“ICA”) and external carotid artery (“ECA”) branches. Each centerline hosts the centers of spheres of maximal radius inscribed in the segmented vessel. In practice, the diameter of a maximally inscribed sphere approximates the minimum diameter of the vessel. These centerline tracts and their associated sphere radii may be used to identify the origin and nominal plane of a bifurcation of the vessel and to split the vessel into its constituent branches. Geometric characterization may then proceed with respect to this vessel-specific coordinate system.

To compute the mutual angles of the branches coming off a bifurcation, branch orientations may first be defined as vectors extending from the branch origins to a point one sphere radius distal. The bifurcation angle may be defined as the angle between the projections of the vectors onto the bifurcation plane.

The measure of vessel tortuosity may be an indicator of vascular abnormalities, and is useful by clinicians for treatment planning (e.g., vessels with high tortuosity may not be suitable targets for stent placement). Vessel tortuosity may be calculated as L/D-1, where L is the length of the centerline from the origin to the end of the branch, and D is the Euclidean distance between these 2 points. Tortuosity may, therefore, be thought of as the fractional increase in length of the tortuous vessel relative to a perfectly straight path. Thus, a tortuosity of 0.0 corresponds to a perfectly straight vessel, whereas a tortuosity of say, 0.2 identifies a vessel 20% longer than the shortest distance between 2 points. Morphological factors may be calculated 17 at this time. A discussion of various morphological factors may be found below.

The method 10 may further comprise the step of storing 19 the calculated geometry of the vasculature, demographic factors, and morphological factors of the individual in the historical vasculature case database. The calculated geometry of the vasculature, demographic factors, and morphological factors may be associated with the individual. For example, this step may be performed by writing the data to an electronic storage device having the case database.

The method 10 further comprises the step of using the processor to identify 21 historical vasculature cases of the case database that resemble the vasculature of the individual. The processor may be configured to perform the identification step 21. For example, the identification 21 step may be performed via software.

The identification step 21 may be performed by the processor based on the calculated geometry of the vasculature and the morphological factors of the historical vasculature cases of the case database. For example, the processor may compare each historical vascular case with one or more of the geometry of the vasculature, the morphological factors, and the demographic factors of the individual. The clinician may weight, or select specific factors to search for (or to exclude from a search). For example, a clinician may limit the comparison to historical vasculature cases where the individual was female. In another example, the clinician may configure the processor to emphasize certain factors like aneurysm size or location. In one embodiment, synthetic descriptors may be generated as search terms. For example, synthetic descriptors may comprise vasculature invariants, geometric characteristics, internal planes of symmetry, etc. The synthetic descriptors may be entirely created by the processor to identify groups of vasculatures with similar characteristics.

In another embodiment, searches may be weighted by the clinician that treated the vasculature. For example, a search algorithm may prioritize certain doctors that are seen as experts in their field.

The clinician may be presented with historical vascular cases that are most similar to the vasculature of the individual. Alternatively, the clinician may be presented with a rupture resemblance scale that indicates the similarity of the individual's vasculature with aneurysms that have ruptured or remained unruptured.

The identification step 21 may also be based on the demographic factors of the individual and the demographic factors of the historical vasculature cases of the case database. The cases of the case database may be identified for the purpose of clinical intervention and/or the purpose of building a rupture resemblance scale database. The historical vasculature cases of the case database may be analyzed by the processor to create a mathematical model to easily predict similarity. The model may be computed before a comparison and after the vasculature of the individual is added to the case database.

In one embodiment, the method 10 further comprises the step of using the processor to calculate 23, for each identified historical vasculature case a morphological resemblance value and a demographic resemblance value. The morphological resemblance value is based on the calculated geometry of the vasculature and the identified historical vasculature case. The demographic resemblance value is based on the demographic factors of the individual and the identified historical vasculature case. These values may be scaled to be in a percentage scale or a number between 0 and 1. The resemblance value may be for each vasculature or the entire corpus of vasculatures. (or another suitable scale). The values may be combined into a total value. The total value may be weighted by a predetermined algorithm, by selection of weights by an artificial neural network, or specifically configured by the clinician.

In another embodiment, the method 10 further comprises the step of determining 25, 27 an aneurysm rupture resemblance scale or aneurysm rupture risk for the vasculature of the individual based on the calculated morphological resemblance values and calculated demographic resemblance values.

There arc important distinctions between aneurysm rupture resemblance scale (“RRS”) and aneurysm rupture risk. An aneurysm rupture risk attempts to predict the probability of an aneurysm rupture in the future, whereas RRS is used to identify aneurysms that highly resemble ruptured aneurysms. In other words, the higher the RRS, the higher the resemblance of an IA is to the ruptured lAs in the case database. RRS uses false-positive cases (unruptured aneurysms classified as ruptured to improve the prediction model. RRS may be valuable for identifying potentially dangerous unruptured aneurysms that highly resemble ruptured aneurysms hemodynamically and/or morphologically. The application of RRS to unruptured aneurysms may provide additional insight for treatment decisions.

The aneurysm rupture resemblance scale, as discussed above, may indicate the resemblance between the individual's vasculature and the vasculatures in the case database. The aneurysm resemblance scale may also indicate the resemblance between the individual's vasculature and the corpus of historical vasculature cases that ruptured (or conversely, remained unruptured). The aneurysm resemblance scale may be useful for clinicians that want to plan for the treatment of an aneurysm using the historical vasculature data, because the clinician can see the outcome of intervention on similar aneurysms.

The aneurysm rupture risk may identify a percentage risk prediction of whether or not the aneurysm will rupture in the future. The aneurysm rupture risk may be based on morphologic, hemodynamic, and demographic factors. The aneurysm rupture risk may be useful to a clinician that needs to make a rapid determination about the urgency of treating an unruptured aneurysm. Both the aneurysm rupture risk and the aneurysm resemblance scale may be presented to the clinician at the same time.

In one embodiment, the method 10 further comprises the steps of using the input device to adjust 39 the geometry of the vasculature and re-calculating 41 the calculated geometry of the vasculature. The geometry of the vasculature may be adjusted 39 to account for the changes in geometry to future clinical intervention and/or the placement of a future surgical device. For example, the clinician may use the input device to simulate placing a coil, surgical stent, flow diverter, or other medical device into the vasculature. By virtue of placing the simulated device, the geometry of the vasculature (with the placed surgical device) may be re-calculated. This may allow the clinician to determine the degree of success a planned intervention may have. The processor may then re-identify historical vasculature cases of the case database that are similar to the virtually modified vasculature. In one embodiment, the processor may suggest the placement of a selected surgical device based on rough simulations or based on historical vasculature cases in the case database.

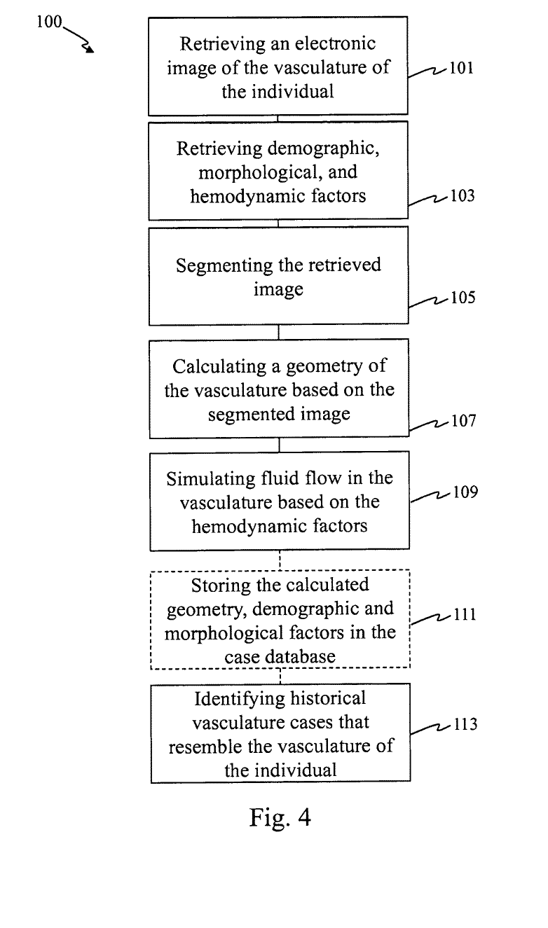

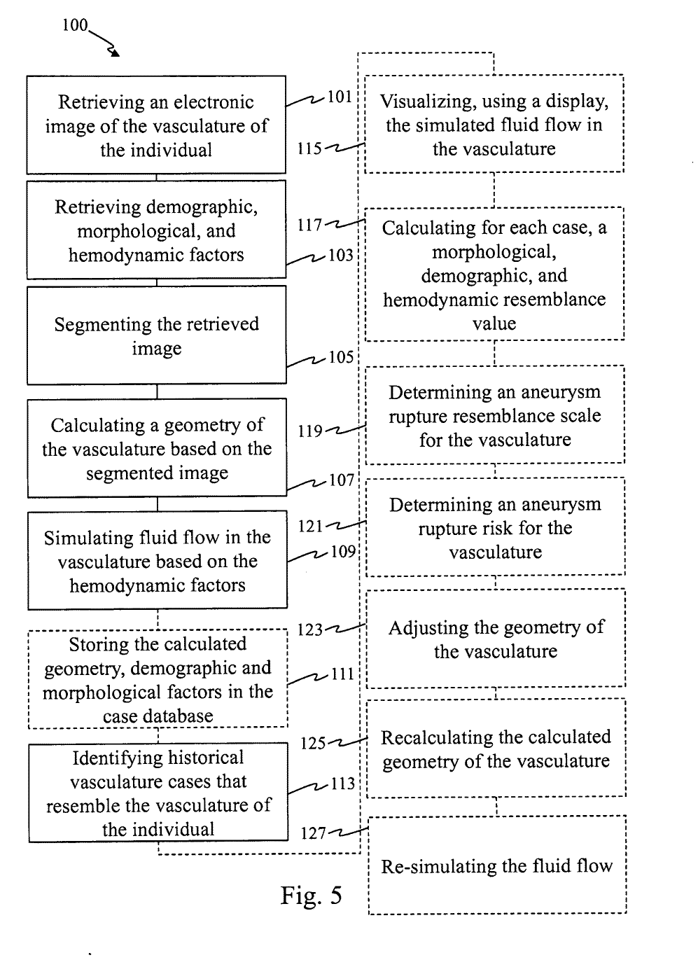

The invention may also be described as a method 100 of identifying historical vasculature cases that resemble a vasculature of an individual, wherein hemodynamics are analyzed. One such method is illustrated in FIGS. 4-5. Hemodynamics is defined as the study of blood flow or circulation. When applied, hemodynamics attempts to explain the physical laws that govern the flow of blood in the blood vessels. These methods 100 use a case database similar to the case database described above except that the historical vasculature cases also comprise hemodynamic factors of the vasculature. Hemodynamic factors may include computational fluid dynamics (“CFD”) results, simulated fluid flow, etc.

The method 100 may comprise retrieving 101 an electronic image of the vasculature of the individual as discussed above. The method 100 may also comprise retrieving 103 demographic factors, morphological factors, and hemodynamic factors of the individual. Hemodynamic factors may include viscosity of the blood and blood density values of the individual. Other hemodynamic factors are described below in detail.

The method 100 further comprises the step of segmenting 105, using the processor, the retrieved image. A geometry of the vasculature is calculated 107 based on the segmented image. The method 100 further comprises the step of using the processor to simulate 109 fluid flow in the vasculature based on the hemodynamic factors associated with the individual and the calculated geometry of the vasculature. In one embodiment, the accuracy and/or speed of simulating 109 fluid flow in the vasculature can be configured using an input device. The simulation step may be performed using a computational fluid dynamics (“CFD”) solver. One example of a CFD solver is GNUID. The solver may rely on a program module that provides a framework for the numerical simulation of partial differential equations. Such a solver can support adaptive mesh refinement computations. Adaptive mesh refinement allows for the changing of accuracy in a solution in certain regions while the solution is being calculated. For example, adaptive mesh refinement may change the spacing of mesh points to change how accurately the solution is known in that region.

The method 100 further comprises the step of storing 111 the calculated geometry of the vasculature, simulated fluid flow of the vasculature, demographic factors, morphological factors, and hemodynamic factors of the individual in the historical vasculature case database.

The method 100 further comprises using the processor to identify 113 historical vasculature cases of the case database that resemble the vasculature of the individual. The identification may be based on the morphological factors of the historical vasculature cases of the case database. The identification 113 may also be based on the simulated fluid flow in the vasculature and/or the hemodynamic factors of the historical vasculature cases of the case database.

In one embodiment, the method 100 further comprises the step of using a display to visualize 115 the simulated fluid flow in the vasculature. For example, the visualized 115 simulated fluid flow may show flow properties as streamlines, streaklines, and pathlines. The flow can either be given in a finite representation or as a smooth function.

In another embodiment, the method 100 further comprises the step of using the processor to calculate 117, for each identified historical vasculature case a hemodynamic resemblance value. The hemodynamic resemblance value may be based on the simulated fluid flow and/or hemodynamic factors in the vasculature and the identified historical vasculature case. An aneurysm rupture resemblance scale for the vasculature may be determined 119. An aneurysm rupture risk for the vasculature may also be determined 121.

In one embodiment, the method may further comprise the steps of adjusting 123 the geometrical parameters of the vasculature, re-calculating 125 the calculated geometry of the vasculature of the individual, and re-simulating 127 the fluid flow in the vasculature based on the re-calculated geometry of the vasculature.

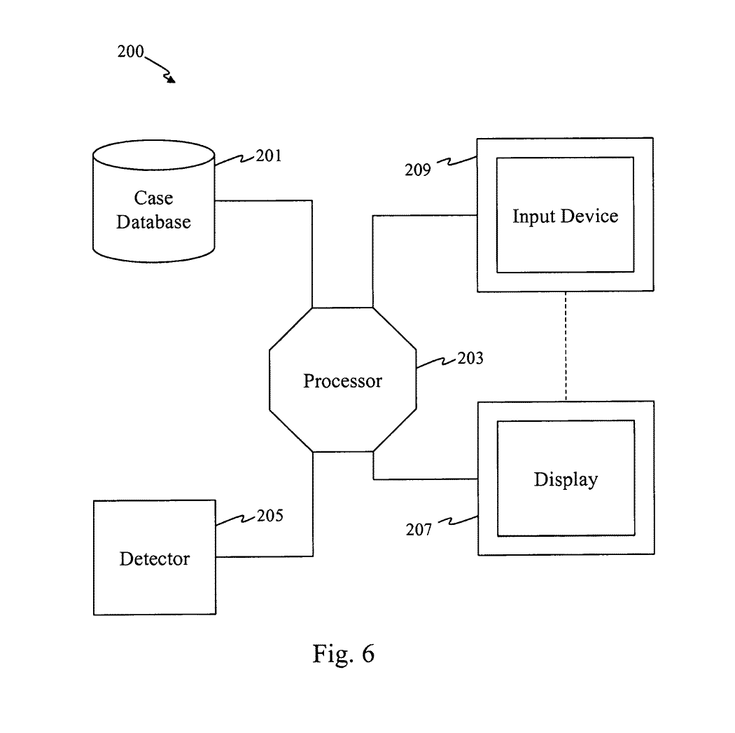

The present invention may also be described as a system 200 for identifying historical vasculature cases that resemble a vasculature of an individual. One such embodiment of system 200 is illustrated in FIG. 6. The system 200 comprises an input device 209, a case database 201, and a processor 203. The input device 209 is configured to capture demographic factors and morphological factors of the individual.

The processor 203 is in electronic communication with the case database 201 and configured to retrieve an image of the vasculature of the individual, retrieve demographic factors, morphological factors, and hemodynamic factors of the individual, segment the retrieved image, calculate a geometry of the vasculature based on the segmented image, store the calculated geometry of the vasculature, demographic factors and morphological factors of the individual in the historical vasculature case database 201, and identify historical vasculature cases of the case database that resemble the vasculature of the individual. The identification may be based on the calculated geometry of the vasculature and the historical vasculature cases of the case database 201 and the demographic factors of the individual and the historical vasculature cases of the case database 201.

In one embodiment, the system 200 further comprises a detector 205 configured to capture an image of vasculature of the individual. The detector 205 may be in electronic communication with the case database 201 and the processor 203. The detector 205 may be configured for digital subtraction angiography, computed tomography angiography, ultrasound, magnetic resonance angiography, and/or any other suitable imaging modality.

In another embodiment, the processor 203 is further configured to simulate fluid flow in the vasculature based on the hemodynamic factors associated with the individual and the calculated geometry of the vasculature and identify historical vasculature cases of the case database 201 that resemble the vasculature of the individual based on the simulated fluid flow in the vasculature and the historical vasculature cases of the case database 201.

In one embodiment, the demographic factors may comprise an age, sex, race, education, blood pressure, health behavior, and/or family history of an individual. Other suitable demographic factors will be apparent in light of the present disclosure. The morphological factors may comprise size, aneurysm height, aneurysm perpendicular height, aspect ratio, bottleneck factor, size ratio, aneurysm inclination angle, aneurysm volume to ostium area ratio, height to width ratio, undulation index, ellipticity index, non-spehericty index, and/or ostium ratio of an individual. Other suitable morphological factors will be apparent in light of the present disclosure. The hemodynamic factors may comprise wall shear stress, oscillatory shear index, energy loss, pressure, pathlines, streaklines, streamlines, particle paths, inflow jet, impingement zone, and/or velocity. Other suitable hemodynamic factors will be apparent in light of the present disclosure. Additional demographic, morphological, and hemodynamic factors are discussed below.

In another embodiment, the system further comprises a display 207 configured to simultaneously show two or more visualizations. The visualizations may both be of the vasculature of the individual. In another embodiment, one visualization may be of a vasculature from the case database 201. The visualization settings may be configured, for example, by a clinician.

In one embodiment, the present invention may be described as a clinician-oriented tool intended for use in routine clinical management of aneurysms. In this embodiment, it may be advantageous that the analysis be performed with less than half an hour of user-interaction time (excluding CFD computation and a printable summary report on the morphology, hemodynamics indices and rupture assessment, and patient characteristics is generated. It may also be advantageous that a built-in mesh generator and CFD solver is incorporated into the tool for ease of use and better workflow.

Exemplary Software Structure

In one embodiment, the processor of the systems executes software to perform the methods of the present invention. An exemplary software embodiment may be designed as a desktop application that interacts with a DICOM viewer through a dedicated plugin. For example, the desktop plugin may be configured to run on Mac OSX and interact with the OsiriX DICOM Viewer. The DICOM viewer may provide connectivity to an institutional PACS. The software may be configured as an integrated tool that embeds imaging and morphological analysis, mesh generation, and post-processing capabilities by leveraging the Vascular Modeling Toolkit (“VMTK”). In addition the software may embed a CFD solver, such as GNUID.

The software may comprise multiple modules, each module having one or more functions. For example, the software may have a meta-data module that collects clinical demographic data. The demographic data may be collected in an anonymized form. The software may have a segmentation module that imports DICOM format images from digital subtraction angiography, computed tomography angiography, and magnetic resonance angiography. Image segmentation may be performed by the module on a branch-by-branch basis as well as segmenting the aneurysm dome. Segmentation may take place by placing seed spheres along the branch of interest on a MIP or on slices and by identifying the corresponding branch name from a pre-defined set. From the seeds, the module initializes a deformable tubular model and makes it evolve towards the lumen boundaries.

The software may also have a morphology analysis module that performs semi-automated computations of morphological indices through the interactive specification of a neckline on a projection image and the selection of the main inlet parent vessel. Indices may be updated on the fly upon selection of different neck lines and inlet vessels.

The software may also have a simulation module that performs mesh generation using the strategy available in VMTK, which in turn may leverage a mesh generator (such as TetGen) for volumetric mesh generation. VMTK may also be used for mesh generation. Users may specify boundary conditions at inlets and outlets from a set of pre-defined or personalized waveforms as well as simulation parameters (viscosity, density, etc.), start a simulation and monitor its progress over time.

The software may also have a CFD solver module. The CFD software module may leverage parallel processing to provide incompressible flow simulation using GNUID. The solver may rely on the libMesh library. Simulations may be transparently managed by the software, and simulation data automatically imported back in the software application for post-processing.

The software may also have a post-processing module for the visualization of bulk hemodynamics. The visualization can be obtained by using pathlines, streaklines, flow jet, and velocity vectors. Visualization of wall hemodynamics can be shown by wall maps of wall shear stress (“WSS”) magnitude, vector in time, and oscillatory shear index (“OSI”) distribution. Synthetic hemodynamic factors relative to the aneurysm sac may be automatically computed and reported to the clinician.

The software may also comprise a report module configured to output a printable report with a summary of the computed hemodynamic and morphologic quantities and assessments.

The software data structure may make use of Bunjee, a Cocoa application framework for medical imaging and visualization applications. Data may be stored as items of a Bunjee case. Items within a Bunjee case may be organized into logical folders. Each case in the case database may be associated with an individual image dataset and can host simulations on multiple aneurysms.

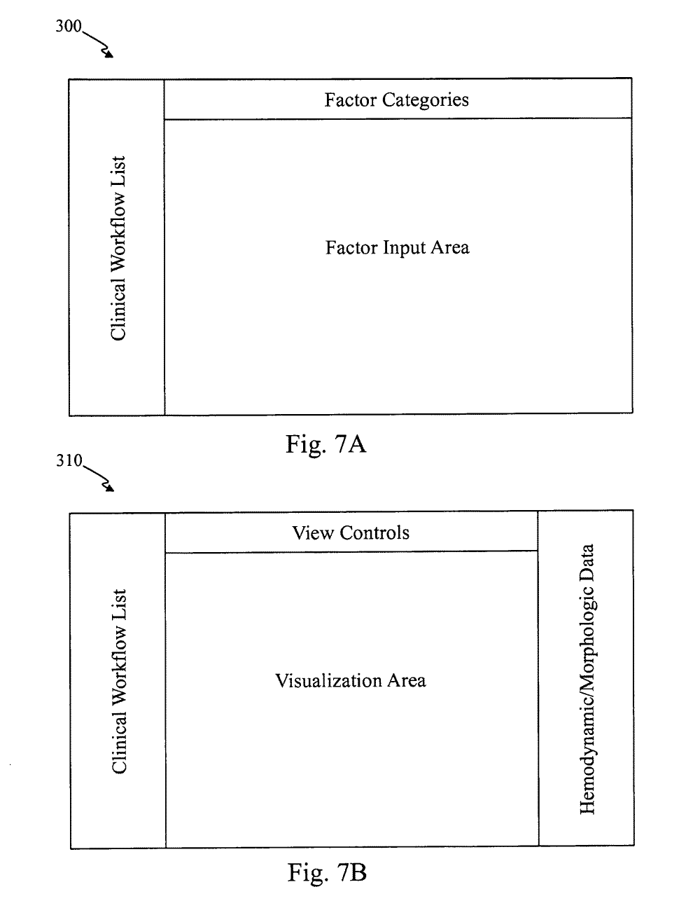

In one embodiment the software has an integrated linear workflow which is designed to be used in a clinical setting used to analyze patient-specific aneurysms. FIGS. 7A and 7B illustrate graphical user interfaces 300, 310 in which the clinician can interact with the software. Graphical user interface 300 illustrates an interface for inputting factors of the individual, such as demographic factors. A list of factor categories may be provided to the clinician and displayed in the factor input area. The factor input area may allow the clinician to enter these factors using a keyboard or mouse. The clinical workflow list may illustrate the current step of the clinician throughout the workflow. The clinical workflow list may allow the clinician to jump to different steps of the workflow at any time, for example, to input additional factors.

FIG. 7B illustrates a graphical user interface 310 that allows a clinician to visualize the vasculature of the individual or the vasculature of a historical vasculature case. Hemodynamic and morphologic data may be displayed to the clinician. The clinician may also be presented with view controls to allow the manipulation of the visualized vasculature.

Meta-data collection, 3D image segmentation, geometrical parameter calculation, meshing, flow simulation setting, and flow result visualization may all be incorporated in a single program designed to analyze aneurysms. The software may be configured to read many different image modalities including Digital Subtraction Angiography, Computed Tomography Angiography, Ultrasound, and Magnetic Resonance Angiography.

The software may assess aneurysms using the demographic factors, morphological (geometric) factors, and hemodynamic (flow simulation) factors. The assessment may be characterized as a similarity to historical ruptured cases. The factors may be compared to a retrospective database of ruptured and unruptured cases. The more similar the current aneurysm is to a ruptured case, the more relevant the historical cases are to the clinicians (and the better a prediction of eventual rupture).

Morphometric parameters may be extracted automatically, however, the user may define the aneurysm neck plane by drawing a line (straight or curved) across the neck plane. The software may allow many different visualizations (images, geometry, mesh, flow results, etc.) that can be viewed at the same time (e.g., in 2×2 panels).

In another embodiment, the software may be configured to use a web-based platform. This allows clinicians to operate the software on a variety of platforms, i.e., Windows/Mac/Linux, workstations/laptops, and portable devices. In one embodiment, the software may leverage cloud computing to speed up the flow computation to provide results in tens of minutes.

Exemplary Morphological Factors for Intracranial Aneurysms

In one embodiment, morphological factors may be directed to factors relevant to intracranial aneurysms. Morphologic factors may include size (the maximum perpendicular height of the IA, i.e., the maximum perpendicular distance of the dome from the neck plane), aspect ratio (“AR”, the ratio of the maximum perpendicular height to the average neck diameter, where the average neck diameter was calculated as twice the average distance from the neck centroid to the edge of the neck), undulation index (1−(V/V_{ch}), where V is the volume of the aneurysm above the neck plane and V_{ch} is the volume of the convex hull). The convex hull of the IA is the smallest volume that fully encloses the IA volume and that is convex at all points.

Morphological factors may include one-dimensional measurements such as aneurysm size (aneurysm perpendicular height), aneurysm maximal height, aneurysm maximal dimension, aneurysm maximal width, neck diameter, and parent vessel diameter. Morphological factors may include two-dimensional measurements such as size ratio, aspect ratio, height-to-width ratio, bottleneck factor, and ostium ratio. Morphological factors may include area or volume measurements such as aneurysm sac surface aneurysm, aneurysm sac volume, aneurysm convex hull surface area, aneurysm convex hull volume. Morphological factors may also include shape indices, such as undulation index, ellipticity index, non-sphericity index, and aneurysm volume-to-ostium area ratio. Morphological factors may also include angle measurements such as aneurysm (inclination) angle, inflow angle, vessel angle, and parent-daugheter angle.

The undulation index (“UI”) captures the degree of IA surface concavity, which can be significant when the aneurysmal sac has strong undulations or when daughter aneurysms are present. The UI increases with the number and severity of concave regions on the IA surface. Conversely, a shape that is nonconcave (e.g., a perfect sphere or a cube) will have a UI of 0.

Another morphologic factor may be ellipticity index (“EI”). The EI characterizes the deviation of the IA convex hull from that of a perfect hemisphere, and is thus a measure of IA elongation. It is defined as EI=1−(18π)^{1/3} V_{ch2/3}/S_{ch}, where S_{ch} is the surface area of the convex hull. Based on the convex hull, the EI is independent of undulations. In contrast to AR, which uses one-dimensional lengths, the EI characterizes IA elongation based on 3D variables such as volume and surface area.

Another morphologic factor may be nonsphericity index (“NSI”). The NSI is similar to the EI, but it uses the actual aneurysm volume and surface area to characterize the deviation of the IA geometry from that of a perfect hemisphere. It is defined as NSI=1−(18π)^{1/3} V^{2/3}/S, where S is the IA surface area. This parameter is influenced by ellipticity and surface undulations and hence can be thought of as a combination of EI and UI.

Another morphologic factor may be size ratio (“SR”). The aneurysm-to-vessel size ratio incorporates the geometries of the IA and its parent vessel and was defined as SR=(maximum aneurysm height)/(average vessel diameter). Here, the average vessel diameter (“DV”) may be obtained by measuring two representative vessel cross sections upstream of the aneurysm (D1 at the proximal neck and D2 at 1.5×D1 upstream), calculating the local diameters in the same way as the neck diameter, and taking their average value. The maximum height in the above equation is not the maximum perpendicular height (H) used in the calculation of size and AR. Rather, it is the maximum (not necessarily perpendicular) distance from the centroid of the aneurysm neck to any point on the aneurysm dome (Hmax). Thus, SR captures the maximum deformation to the parent vessel caused by the outpouching of an IA. In the case of a terminal aneurysm, the IA bulges out from several vessels. Therefore, the average diameter of the feeding and all branching vessels was used for the “average vessel diameter” in this case.

SR takes into account not only the aneurysm size itself but also the local vessel caliber and incorporates it into a quantifiable parameter. By doing so, it indirectly accounts for the effect of IA location on rupture. A particularly high rupture risk exists in the anterior regions of the circle of Willis, and a large percentage of small aneurysms that rupture are located on the anterior communicating artery (AComA). In contrast to the high rupture incidence in the anterior circulation, it was also found that IAs on the cavernous part of the internal carotid artery (ICA) and on the ophthalmic artery seldom rupture. In general, vessel size and wall thickness decrease behind branching points, and the more distally an IA is located in the arterial tree, the smaller its wall thickness and parent vessel diameter will be. The fact that lAs rupture most often on the AComA and least on the ICA may be at least partly accounted for by SR.

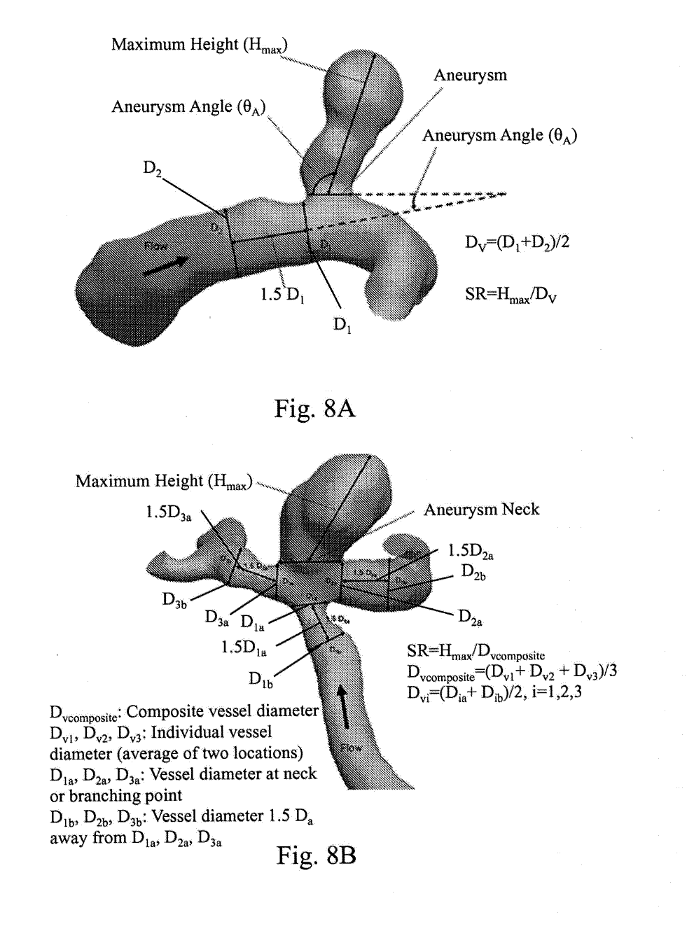

Another morphologic factor may be vessel angle. FIGS. 8A and 8B illustrate one method of calculating vessel angle, among other morphological factors. Apart from the aneurysm geometry, the parent vessel inlet angle relative to the aneurysm has been shown to have a large effect on intraaneurysmal flow. The vessel inlet angle or simply, the vessel angle (“θᵥ”), was defined as the angle between the inlet vessel centerline and the neck plane. The centerline is approximated by the line connecting the centroids of the two vessel cross sections used to define “average vessel diameter” in SR. It should be noted that Ov is only defined for sidewall lAs. Because the value of Ov depends on the direction from which the geometry is viewed, a rigorous definition of the viewing plane is necessary.

Another morphologic factor may be aneurysm inclination angle. Because IA hemodynamics will also be influenced by the angle at which the IA is tilted with respect to the incoming flow, the aneurysm inclination angle (θ_{A}) may be defined as another morphological factor. This is the angle of inclination between the IA and its neck plane. The line inside the IA used for defining θ_{A} is the connection of the neck centroid to the farthest point on the IA dome. Aneurysms with daughter aneurysms and secondary growth are expected to have a higher value of this angle. As with θᵥ, the θ_{A} is only defined for sidewall IAs and depends on the direction from which the geometry is viewed. The association of larger aneurysm angles with higher rupture risk may be linked to the presence of daughter aneurysms (or blebs on the main aneurysm sac), which is known to be correlated with increased IA rupture risk. Blebs are usually located on the dome side opposed to incoming flow, causing a higher aneurysm angle. In addition, the highest rates of IA growth typically occur in the regions of lowest WSS (located at the far end of the dome), whereas the main direction of IA growth was aligned with the direction of the incoming flow. Thus, a greater θ_{A} with respect to the parent vessel could be caused either by the presence of a daughter aneurysm/bleb, or by increased growth of the IA in the direction of the flow.

The viewing plane used for the definition of θᵥ and θ_{A} may be chosen so that it captures the direction of the incoming flow entering a side-wall IA. In other words, the velocity vectors of the flow immediately upstream of the IA should lie inside the viewing plane. Most geometries in individuals have vessels that bend in all three dimensions, and it is essential to view the geometry from a direction at which that incoming flow is visible. The correct viewing plane may be determined by rotating, the geometry until the neck plane is seen as a line. That means that the IA neck plane must be perpendicular to the viewing plane. Then, an axis of rotation through the neck centroid is defined perpendicularly to the neck plane, and the entire geometry is rotated about this axis. The view that results in the lowest value of the apparent vessel angle (apparent θᵥ) is chosen to be the viewing plane in which θᵥ and θ_{A} are finally measured. Even though θ_{A}, at first glance, seems to have no relation with the parent vessel, it is important to note that θ_{A} is just as dependent on the viewing plane as Ov. If the viewing plane was chosen differently, the measured θ_{A} would also be different.

A limitation of previous morphology-based rupture risk studies, including those investigating 3D parameters, is that the geometry of the parent artery is typically ignored. Parent artery geometry has a significant influence on the resultant IA hemodynamics and, consequently, the rupture risk. Upstream vessel tortuosity can critically influence intra-aneurysmal hemodynamics. A greater parent vessel incidence angle shifts the high WSS area toward the aneurysm dome, where rupture-prone blebs often are present. Highly curved parent vessels subject IAs to higher hemodynamic stresses at the inflow zone that might promote growth or rupture. Thus, parent vessel geometry should be accounted for when defining morphological factors for IAs. Furthermore, there may be a connection between IA rupture risk and vessel location. Because vessel location is strongly related to vessel geometry, this finding affirms the importance of vessel geometry for IA rupture risk. Incorporating parent vessel geometry in morphology factors can, at least to some extent, capture the influence of IA location as well.

Exemplary Demographic Factors for Intracranial Aneurysms

In one embodiment, demographic factors may be directed to factors relevant to intracranial aneurysms. Demographic factors may include socioeconomic features (e.g., age, gender, race, and education level). For example, the age may be presented to the clinician and stored in the case database in ranges (i.e., 10-19, 20-29, etc.) Race and education level may be presented to the clinician and stored in the case database in pre-determined categories. These factors may also be stored as text, thereby allowing the clinician to enter custom information for these and other factors.

Demographic factors may also include the medical history of the individual. For example, the demographic factors may include symptomatic medical history (e.g., nausea or vomiting, stiff neck or neck pain, blurred or double vision, pain above and behind eye, dilated pupils, sensitivity to light, loss of sensation, peripheral vision deficits, thinking or processing problems, speech complications, perceptual problems, sudden changes in behavior, loss of balance and coordination, decreased concentration, short-term memory difficulty, and fatigue). The demographic factors may also include any history of intracranial aneurysms (e.g., ruptured or unruptured intracranial aneurysms, location, and notes). The demographic factors may also include metabolic history (e.g., history of hypertension, coronary artery disease, peripheral vascular disease, diabetes, high body mass index, and hyperlipidermia). The demographic factors may include other medical history such as menopausal status, thyroid disease, and polycystic kidney disease.

Demographic factors may also include family history of the individual or health behaviors of the individual. For example, family history may include occurrences of subarachnoid hemorrhages or intracranial aneurysms in family members. Other history factors may include Ehlers Danlos Syndrome, aortic coarctation, intracranial arteriovenous malformation, and carotid stenosis or occlusion. Health behaviors may include tobacco intake (e.g., current smoker, former smoker, packs/day, years of smoking, duration after quitting), alcohol intake (e.g., alcohol abuse markers, drinks per day), caffeinated drink intake (e.g., average intake per day), and drug exposure (e.g., exposure to cocaine, marijuana, aspiring, phenylpropanolamine, anticoagulants, and oral contraceptives).

Demographic factors may also include data on historical intracranial aneurysms. For example this data may include the site of historical aneurysms in the individual, the type of aneurysm (e.g., saccular, fusiform, dissecting, terminal), and whether or not the aneurysm ruptured. Since site descriptions may not be consistent among treatment centers, the associated fields may be restricted to certain sites (e.g., ICA Terminus, ICA-ophthalmic, ICA-anterior choroidal, ICA-PcomA, other ICA, AcomA, MCA (primary branches), MCA (secondary branches), pericallosal-callosomarginal, basilar terminus, basilar trunk, vertebrobasilar junction, VA-PICA, BA-SCA, and BA-PCA). The demographic factors may also include information on the individual's existing treatment plans, their history of ischemic stroke, and whether they have an incomplete (atypical) circle of Willis (e.g., incompleteness of Al segments of ACA, asymmetry of Al segments of ACA, incompleteness of P1 segments of PCA, asymmetry of P1 segments of PCA, incompleteness of PcomA ipsilaterla, P1 segment of PCA>PcomA ipsilateral, PcomA>P1 segment of PCA ipsilateral). In cases where the individual was previously treated for an intracranial aneurysm, the treatment method (e.g., clip, coil, stent assisted coiling, flow diverter, etc.) may be included as well as the number of aneurysms previously detected. Outcomes and complications of historical aneurysms may be included in the demographic factors (e.g., intraprocedural rupture, morbidity due to intraprocedural rupture, mortality due to intraprocedural rupture, morbidity due to thromboembolic complications, postoperative complete occlusion, postoperative incomplete occlusion, postoperative failed occlusion, disability rank, RANKIN scale).

Exemplary Computational Fluid Dynamics Modeling

In one embodiment, each IA model was meshed using a mesh generator (such as TetGen) to create finite volume tetrahedral elements and wall prism elements (for accurate boundary layer resolution). The incompressible Navier-Stokes equations may be solved numerically under pulsatile flow conditions using CFD solvers, such as GNUID. Published or patient-specific mean flow rates may be used as inlet boundary conditions for given vessel locations. A pulsatile velocity waveform may be obtained from transcranial doppler ultrasound or phase-contrast magnetic resonance measurement on a normal subject or patient-specific subject with its magnitude scaled to the desired mean flow rate. Traction-free boundary conditions may be implemented at the outlet. A mass flow rate through each outlet artery may be calculated to be proportional to the cube of its diameter based on the principle of optimal work. Because the non-Newtonian effect is generally negligible in medium and large arteries (those harboring aneurysms), blood may be modeled as a Newtonian fluid with a density of 1,056 kg/m³ and a viscosity of 0.0035 N*s/m².

Three pulsatile cycles may be simulated to ensure that numeric stability had been reached, and the last cycle may be taken as output. In one embodiment, hemodynamic data may be presented as time averages over the third pulsatile cycle of flow simulation.

Hemodynamic Factors for Intracranial Aneurysms

In one embodiment, some hemodynamic factors may be based on simulated fluid flow in the aneurysm. Some hemodynamic factors may include WSS, maximum intra-aneurysmal WSS (MWWS), low WSS area, WSS gradient (WSSG), oscillatory shear index (“OSI”), number of vortices (NV), and relative residence time (RTT).

Hemodynamic factors may include qualitative aneurysmal flow-based factors such as streamlines, pathlines, streaklines, particle paths, inflow jet, impingement zone, and vortex coreline. Hemodynamic factors may also include quantitative aneurysmal flow-based factors such as velocity, inflow concentration index, number of vortices, and vortex length. Hemodynamic factors may include wall shear stress-based factors and may be defined as time average/systolic peak/end of diastole, spatial average/minimum/maximum, or normalized by parent vessel value. Such factors may include wall shear stress, low shear-stress area percentage, low shear index, shear concentration index, oscillatory shear index, relative residence time, wall shear stress gradient, and gradient oscillatory number. Hemodynamic factors may also include energy-based factors such as pressure loss, energy loss, pressure loss coefficient, kinetic energy ratio, and viscous dissipation ratio.

The concept of WSS refers to the tangential, frictional stress caused by the action of blood flow on the vessel wall. For pulsatile flow, the time-averaged WSS may be calculated by integrating the WSS magnitude at each node over the cardiac cycle:

$$WSS = \frac{1}{T} {\int }_{0}^{T} \vert {wss}_{i} \vert d t , \qquad ( Eq . 1 )$$

where wssᵢ is the instantaneous shear stress vector and T is the duration of the cycle. In one embodiment, WSS distributions may be normalized by the average parent vessel WSS in the same individual to allow comparison among different individuals. Hemodynamic factors may also include WSS-based parameters, such as: (1) WSS, defined as WSS (already time-averaged, as shown in Eq. 1), further averaged over the dome area (the entire luminal surface of the aneurysm sac); (2) MWSS, defined as maximum intra-aneurysmal WSS magnitude normalized by the parent vessel WSS; (3) low WSS area (LSA), defined as the areas of the aneurysm wall exposed to a WSS below 10% of the mean parent arterial WSS and then normalized by the dome area; (4) WSSG, measuring the change of WSS magnitude in the flow direction and calculated by taking the spatial derivative of WSS with respect to the streamwise distance. In one embodiment, the time-averaged WSSG may be further averaged over the dome area.

OSI, a nondimensional parameter, measures the directional change of WSS during the cardiac cycle.

$$OSI = \frac{1}{2} \{ 1 - \frac{\vert {\int }_{0}^{T} {wss}_{i} d t \vert}{{\int }_{0}^{T} \vert {wss}_{i} d t \vert} \} , \qquad ( Eq . 2 )$$

where wssᵢ is the instantaneous WSS vector and T is the duration of the cycle. OSI is often used to describe the disturbance of a flow field. OSI is defined as OSI averaged over the dome area.

NV may be counted based on the velocity field of the representative cross-sectional plane for each aneurysm.

A combination of WSS and OSI reflects the residence time of blood near the wall. RRT quantifies the state of disturbed flow. By incorporating Eq. 1 and Eq. 2 into the definition, RRT is inversely proportional to the magnitude of the time-averaged WSS vector:

$$RRT = \frac{1}{( 1 - 2 xOSI ) \times WSS} = \frac{1}{\frac{1}{T} \vert {\int }_{0}^{T} {wss}_{i} d t \vert} \qquad ( Eq . 3 )$$

Although they are analyzed by different modalities (e.g., 3-dimensional angiography and CFD, respectively), morphology and flow dynamics are intricately related to each other. Low WSS and high OSI—the flow characteristics significantly associated with rupture—are common in aneurysms of complex or elongated shapes described by large SR, aspect ratio, ellipticity index, and UI values. These morphological features have been shown in association with growth and rupture. Furthermore, as the SR value exceeds 2, the single aneurismal vortex splits into multiple vortices and LSA increases drastically in both sidewall and terminal aneurysm models.

The relationship between morphology and hemodynamics may be complex, interwoven, and dynamic. From an aneurysm evolution point of view, mutually contributive roles of “unfavorable” aneurysm morphology and “unfavorable” aneurysm hemodynamics may be captured. For example, hemodynamics of low WSS-high OSI is conducive for atherosclerotic change and inflammatory responses, which could drive heterogeneous remodeling of the aneurysm wall and aneurysm growth. Such growth could lead to an increasing SR and a more complex shape. Complex geometry and high SR values, in turn, would lead to more complex flow patterns with low and oscillatory WSS.

Exemplary Case Characterization

In one embodiment, each vasculature is characterized to allow for searching and comparison. For example the mean and SDs of all morphological and hemodynamic parameters maybe calculated for ruptured and unruptured groups. A Jarque-Bera test for departure from a normal distribution may be performed to determine if a parameter was normally distributed. A 2-tailed independent Student t test (for normally distributed data) or Wilcoxon rank-sum test (for abnormally distributed data) may be performed for each parameter to assess the statistical significance of the observed difference between the mean values of ruptured and unruptured groups. Probability values from calculated and statistical significance may be assumed for P<0.01. Receiver operating characteristics (ROC) analysis may be performed on all parameters to determine the optimal thresholds separating ruptured and unruptured groups by calculating the area under the ROC curve (AUC).

The parameters found to be significant (P<0.01) may be further analyzed using multivariate logistic regression (backward elimination) to identify those that retained significance when accounting for all relevant variables. Before performing the regression, each variable may be scaled to span a range from 0 to 10, thereby easing OR comparison by ensuring that a unit increase in the parameter corresponded to 10% of its observed range.

Logistic regression may then performed on the significant variables (in the morphological category alone, hemodynamic category alone, demographic alone, and combined categories) to find final parsimonious models that allowed calculation of the quantitative risk of aneurysm rupture. These models may predict the probability of an aneurysm being ruptured on the basis of morphology, hemodynamics, and demographics, or combination thereof. To compare the ability of the individual regression models to discriminate rupture status, the AUC-ROC may be calculated on the predicted probability of rupture status from these regression models. Results may be analyzed for statistically significant differences in the AUC-ROC values of the 3 models.

In one embodiment the fluid flow may be characterized by the processor. For example, hemodynamic simulations may show that most ruptured aneurysms had complex flow patterns with multiple vortices. In contrast, most unruptured aneurysms may have simple flow patterns with a single vortex. On each of the middle cross-sectional plane flow pattern visualizations, the cross-sectional velocity vector plane may be overlapped on top of the corresponding aneurysm geometry to clearly show the cutting plane. In ruptured IAs, WSS values may be lower within the aneurysm than in the parent vessels, whereas in unruptured IAs, they may be comparable. Ruptured aneurysms may have lower WSS magnitudes and larger areas of low WSS than unruptured aneurysms. The distributions of OSI for ruptured and unruptured aneurysms show that ruptured IAs may have a higher OSI than unruptured IAs.

To identify independent parameters having significant correlation with ruptured IA, multivariate logistic regression analysis may be performed separately on significant morphological parameters—SR, UI, ellipticity index, and nonsphericity index—and significant hemodynamic parameters—WSS, MWSS, LSA, OSI, NV, and RRT—and combined significant morphological and hemodynamic parameters. Having regressed for the variables using a backward elimination process, a final parsimonious model may be obtained for discriminating rupture status from the morphology-based model (Odd_{M}) and the hemodynamics-based model (Odd_{H})as well as the combined model (Odd_{Combined}). For example:

Odd_{M}=e^{1.09·SR-2.99} (Eq. 4)

Odd_{H}=e^{-0.73·WSS+2.86·OSI-0.12} (Eq. 5)

Odd_{Combined}=e^{0.73·SR-0.45·WSS+2.19·OSI-2.09} (Eq. 6)

Further to the example, in the morphology-based model (Eq. 4), only SR was retained as an independently significant parameter (AUC, 0.83; 95% CI, 0.75 to 0.91). For a unit increase in the SR of an IA, the odds of IA rupture increased by 2.96 times. In the hemodynamics-based model (Eq. 5), both WSS and OSI were retained as independently significant parameters (AUC, 0.85; 95% CI, 0.78 to 0.93). When the regression was per formed on significant morphological and hemodynamic parameters combined, using the same backward elimination process, the composite model, Eq. 6, consisted of SR, WSS, and OSI (AUC, 0.89; 95% CI, 0.82 to 0.96). SR was inversely correlated with low WSS (with Pearson correlation coefficient of 0.001, R²=−0.53). SR is a relatively simple measure that considers aneurysm maximum height in relation to the parent artery. Thus, SR captures the maximum shape deformation to the parent vessel caused by the outpouching of an aneurysm and intuitively reflects the degree of aneurismal degradation to the vessel. IA location is a strong factor in clinical decision-making (e.g., IAs on the ACOM—a smaller artery—rupture far more frequently than IAs arising from the internal carotid artery). Incorporating the parent vessel caliber in morphology parameters can, at least to some extent, capture the influence of IA location as well.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A method of identifying historical vasculature cases that resemble a vasculature of an individual using a case database comprising historical vasculature cases, and a processor in electronic communication with the case database, the method comprising the steps of:
retrieving an electronic image of the vasculature of the individual;
retrieving demographic factors and morphological factors of the individual;
segmenting the retrieved image, using the processor;
calculating, using the processor, a geometry of the vasculature based on the segmented image; and
identifying, using the processor, historical vasculature cases of the case database that resemble the vasculature of the individual based on:
the calculated geometry of the vasculature and the morphological factors of the historical vasculature cases of the case database; and
the demographic factors of the individual and the demographic factors of the historical vasculature cases of the case database.

2. The method of claim 1, further comprising the step of storing the calculated geometry of the vasculature, demographic factors and morphological factors of the individual in the historical vasculature case database.

3. The method of claim 1, further comprising the step of:
calculating, using the processor, for each identified historical vasculature case:
a morphological resemblance value based on the calculated geometry of the vasculature and the identified historical vasculature case; and
a demographic resemblance value based on the demographic factors of the individual and the identified historical vasculature case.

4. The method of claim 3, further comprising the step of determining an aneurysm rupture resemblance scale for the vasculature of the individual based on the calculated morphological resemblance values and calculated demographic resemblance values.

5. The method of claim 3, further comprising the step of determining an aneurysm rupture risk for the vasculature of the individual based on the calculated morphological resemblance values and calculated demographic resemblance values.

6. The method of claim 1, wherein the step of retrieving an image of the vasculature of the individual comprises the sub-steps of:
capturing, using a detector, an image of the vasculature of the individual; and
storing the image of the vasculature of the individual such that the vascular image is electronically associated with the individual.

7. The method of claim 6, wherein the image of the vasculature is stored in an electronic picture archiving and communication system.

8. The method of claim 6, wherein the image of the vasculature is stored in the case database.

9. The method of claim 1, wherein the step of retrieving demographic factors and morphological factors of the individual comprises the sub-steps of:
inputting, using an input device, the demographic factors and morphological factors of the individual; and
storing the demographic factors and morphological factors of the individual such that the demographic factors and morphological factors are associated with the individual.

10. The method of claim 9, wherein the input device is a laptop, mobile phone, workstation, tablet, and/or a front-end computer.

11. The method of claim 9, wherein the demographic factors, morphological factors, and hemodynamic factors of the individual is stored in the case database.

12. The method of claim 1, further comprising the step of defining, using an input device, a neck plane, region of interest, and/or a main inlet parent vessel.

13. The method of claim 12, wherein the segmenting the retrieved image is based on the defined neck plane, region of interest, and/or main inlet parent vessel.

14. The method of claim 1, further comprising the steps of:
adjusting, using the input device, the geometry of the vasculature; and
re-calculating the calculated geometry of the vasculature.

15. The method of claim 14, wherein the geometry of the vasculature is adjusted to account for future clinical intervention.

16. The method of claim 14, wherein the geometry of the vasculature is adjusted to account for a future surgical device.

17. The method of claim 1, wherein the retrieved image is segmented, using the processor, on a branch-by-branch basis.

18. The method of claim 17, wherein the step of segmenting the retrieved image comprises the sub-steps of:
placing, using an input device, seed spheres along a branch of interest; and
identifying, using the input device, a corresponding branch name.

19. The method of claim 1, wherein the historical vasculature cases of the case database are identified for the purpose of clinical intervention.

20. The method of claim 1, wherein the historical vasculature cases of the case database are identified for the purposes of building a rupture resemblance scale database.

21. A method of identifying historical vasculature cases that resemble a vasculature of an individual using a case database comprising historical vasculature cases, and a processor in electronic communication with the case database, the method comprising the steps of:
retrieving an image of the vasculature of the individual;
retrieving demographic factors, morphological factors, and hemodynamic factors of the individual;
segmenting the retrieved image, using the processor;
calculating, using the processor, a geometry of the vasculature based on the segmented image;
simulating, using the processor, fluid flow in the vasculature based on the hemodynamic factors associated with the individual and the calculated geometry of the vasculature; and
identifying, using the processor, historical vasculature cases of the case database that resemble the vasculature of the individual based on:
the calculated geometry of the vasculature and the morphological factors of the historical vasculature cases of the case database;
the simulated fluid flow in the vasculature and the hemodynamic factors of the historical vasculature cases of the case database; and
the demographic factors of the individual and the demographic factors of the historical vasculature cases of the case database.

22. The method of claim 21, further comprising the step of storing the calculated geometry of the vasculature, simulated fluid flow of the vasculature, demographic factors, morphological factors, and hemodynamic factors of the individual in the historical vasculature case database.

23. The method of claim 21, further comprising the step of visualizing, using a display, the simulated fluid flow in the vasculature.

24. The method of claim 21, wherein the hemodynamic factors include viscosity and density values of the individual.

25. The method of claim 21, wherein the accuracy and/or speed of simulating fluid flow in the vasculature can be configured using an input device.

26. The method of claim 21, further comprising the step of:
calculating, using the processor, for each identified historical vasculature case:
a morphological resemblance value based on the calculated geometry of the vasculature and the identified historical vasculature case;
a demographic resemblance value based on the demographic factors of the individual and the identified historical vasculature case; and
a hemodynamic resemblance value based on the simulated fluid flow in the vasculature and the identified historical vasculature case.

27. The method of claim 26, further comprising the step of determining an aneurysm rupture resemblance scale for the vasculature of the individual based on the calculated hemodynamic resemblance values, calculated morphological resemblance values, and calculated demographic resemblance values.

28. The method of claim 26, further comprising the step of determining an aneurysm rupture risk for the vasculature of the individual based on the calculated hemodynamic resemblance values, calculated morphological resemblance values, and calculated demographic resemblance values.

29. The method of claim 21, further comprising the steps of:
adjusting the geometrical parameters of the vasculature;
re-calculating the calculated geometry of the vasculature of the individual; and
re-simulating the fluid flow in the vasculature based on the re-calculated geometry of the vasculature.

30. A system for identifying historical vasculature cases that resemble a vasculature of an individual comprising:
an input device configured to capture demographic factors and morphological factors of the individual;
a case database in electronic communication with the input device, the case database configured to comprise historical vasculature cases;
a processor in electronic communication with the case database, the processor configured to:
retrieve an image of the vasculature of the individual;
retrieve demographic factors, morphological factors, and hemodynamic factors of the individual;
segment the retrieved image;
calculate a geometry of the vasculature based on the segmented image;
store the calculated geometry of the vasculature, demographic factors and morphological factors of the individual in the historical vasculature case database; and
identify historical vasculature cases of the case database that resemble the vasculature of the individual based on:
the calculated geometry of the vasculature and the historical vasculature cases of the case database; and
the demographic factors of the individual and the historical vasculature cases of the case database.

31. The system of claim 30, further comprising a detector configured to capture an image of vasculature of the individual.

32. The system of claim 30, wherein the processor is further configured to:
simulate fluid flow in the vasculature based on the hemodynamic factors associated with the individual and the calculated geometry of the vasculature; and
identify historical vasculature cases of the case database that resemble the vasculature of the individual based on the simulated fluid flow in the vasculature and the historical vasculature cases of the case database.

33. The system of claim 30, wherein the detector is configured for digital subtraction angiography, computed tomography angiography, ultrasound, and/or magnetic resonance angiography.

34. The system of claim 30, wherein the demographic factors comprise an age, sex, race, education, blood pressure, health behavior, and/or family history of an individual.

35. The system of claim 30, wherein the morphological factors comprise size, aneurysm height, aneurysm perpendicular height, aspect ratio, bottleneck factor, size ratio, aneurysm inclination angle, aneurysm volume to ostium area ratio, height to width ratio, undulation index, ellipticity index, non-spehericty index, and/or ostium ratio of an individual.

36. The system of claim 30 wherein the hemodynamic factors comprise wall shear stress, oscillatory shear index, energy loss, pressure, pathlines, streaklines, streamlines, particle paths, inflow jet, impingement zone, and/or velocity.

37. The system of claim 30, further comprising a display configured to simultaneously show two or more visualizations of the vasculature of the individual.
